(19) United States
(12) Patent Application Publication
(10) Pub. No.: US 2011/0054128 A1
(43) Pub. Date: Mar. 3, 2011

(54) Controlling Prime Olefin Ratio In An Oxygenates-To-Olefins Reaction

(76) Inventors: Guang CAO, Princeton, NJ (US); Thomas H. Colle, Houston, TX (US); Luc R.M. Martens, Meise (BE); Stephen H. Brown, Bernardsville, NJ (US); Teng Xu, Hampton, NJ (US)

(21) Appl. No.: 12/837,546

(22) Filed: Jul. 16, 2010

Related U.S. Application Data
(60) Provisional application No. 61/238,349, filed on Aug. 31, 2009.

(51) Int. Cl. C08F 2/00; C07C 1/00

(52) U.S. Cl. 526/75; 585/324

(57) ABSTRACT

Disclosed herein is a method of controlling production of olefins in an oxygenates-to-olefins reaction by combining in a reactor methanol and a molecular sieve, a AlPO or SAPO in certain embodiments, under conditions to produce at least ethylene and propylene having a first ethylene/propylene ratio; adding to the reactor a first amount of a C₁ to C₅ aldehyde; and withdrawing from the reactor a first amount of ethylene and propylene having a second ethylene/propylene ratio, wherein the second ethylene/propylene ratio is greater than the first ethylene/propylene ratio. The aldehyde is added at the same time, or co-feed, with the methanol under the same reaction conditions.

### CROSS-REFERENCE TO RELATED APPLICATION

This application claims priority to U.S. Provisional Patent Application No. 61/238,349, filed Aug. 31, 2009, the disclosure of which is incorporated herein by reference in its entirety.

### FIELD OF THE INVENTION

This disclosure relates to oxygenate-to-olefins reaction catalyzed by molecular sieves, and more particularly to the control of the ethylene/propylene ratio generated by reacting certain molecular sieves with methanol influenced by the presence of an aldehyde.

### BACKGROUND

Controlling product composition is of great significance in an oxygenate-to-olefins (OTO) process. A variety of means for controlling the reaction are known such as by pre-pooling the molecular sieve or co-feeding co-reactants, but also by varying molecular sieve or catalyst identity. In particular, controlling the structure of the molecular sieve that is used in the reaction is another means of controlling the reaction and affecting the ratio of ethylene and propylene generated therefrom.

The OTO reactor conditions can also be varied to control the ratio of ethylene to propylene, or the Prime Olefin Ratio (“POR”). Higher temperature and lower pressure favor lighter product (ethylene), and higher steady-state coke loading on recirculating molecular sieve also favors ethylene. The term “coke” here and throughout refers to carbon compounds formed in and upon the molecular sieves as a result of its being exposed to oxygenates under catalytic conditions. Pre-pooling (pre-coking of freshly regenerated molecular sieve) has been shown to favor ethylene, and the additions of metal oxide co-molecular sieve favors propylene and higher olefins.

What would be useful is another means to control the oxygenate-to-olefins reaction, and in particular, to control the POR. The inventors describe herein such methods. The inventors demonstrate control of the reaction by increasing the ethylene to propylene ratio while decreasing on-feed lifetime. Disclosed herein is a means of controlling the ratio of ethylene to propylene by effectively using a desirable molecular sieve with a combination of molecular sieve structures (AEI vs. CHA structures) and feed compositions.

Related disclosures include U.S. Pat. No. 7,205,447; U.S. Pat. No. 7,132,581; and U.S. 2006-0149109, each of which is incorporated by reference.

### SUMMARY

Disclosed in one embodiment is a method of controlling production of olefins in an oxygenates-to-olefins reaction comprising, or consisting essentially of, combining in a reactor methanol and a molecular sieve under conditions to produce at least ethylene and propylene having a first ethylene/propylene ratio; adding to the reactor a first amount of a C₁ to C₅ aldehyde, and withdrawing from the reactor a first amount of ethylene and propylene having a second ethylene/propylene ratio, wherein the second ethylene/propylene ratio is greater than the first ethylene/propylene ratio.

In another aspect, disclosed herein is a method of producing ethylene.

In certain embodiments, the addition is performed in one step at the same reactor conditions as the combining step.

In certain other embodiments, the molecular sieve is an aluminophosphate or a silicoaluminophosphate.

In yet other embodiments, the silicoaluminophosphate is selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, metal containing molecular sieves thereof, and combinations thereof.

The various descriptive elements and numerical ranges described can be combined with other descriptive elements and numerical ranges to describe preferred embodiments of the processes disclosed herein; further, any upper numerical limit of an element can be combined with any lower numerical limit of the same element to describe preferred embodiments. In this regard, the phrase “within the range from X to Y” is intended to include within that range the “X” and “Y” values.

Unless otherwise noted, if an amount of a component is stated, that amount is understood to be an aggregate amount if two or more different species of that component are present together.

### DETAILED DESCRIPTION

### Introduction

The inventors have found that certain molecular sieves, under oxygenate-to-olefins conversion conditions, respond to the presence of aldehydes with a change in the ratio of ethylene to propylene, and particularly, an increase in the ethylene to propylene. Thus, described herein is a method of controlling production of olefins in an oxygenates-to-olefins reaction that comprises combining in a reactor oxygenates, including at least methanol, and a molecular sieve under conditions to produce at least ethylene and propylene having a first ethylene/propylene ratio, adding to the reactor a first amount of a C₁ to C₅ aldehyde, and withdrawing from the reactor a first amount of ethylene and propylene having a second ethylene/propylene ratio, wherein the second ethylene/propylene ratio is greater than the first ethylene/propylene ratio.

Of course, the amount of aldehyde can be increased, resulting in a further increase in the ethylene/propylene ratio, or Prime Olefin Ratio (“POR”). So, for example, in another embodiment, a third amount of a C₁ to C₅ aldehyde is added to the reactor, wherein the third amount of aldehyde is greater than the first amount of aldehyde; withdrawing from the reactor a second amount of ethylene and propylene having a second ethylene/propylene ratio, wherein the second ethylene/propylene ratio is greater than the first ethylene/propylene ratio, or greater POR.

The aldehyde, when added, is combined with the molecular sieve at the same time as the oxygenates are combined with the same molecular sieve. There is no pretreatment of the molecular sieve with the aldehyde, so, desirably, the aldehydes are added in one step to produce olefins with the increased POR. The typical disengaging and/or regeneration process is used to recycle the molecular sieve back to the reactor for further catalysis. Preferably, when the aldehyde is combined with the oxygenates and molecular sieve, it is co-fed with the oxygenates at the same conditions used when aldehyde is absent and only oxygenates, such as methanol, is added to the molecular sieve. Thus, in a particular embodiment, the methanol, molecular sieve and aldehyde are combined in one or more riser reactor(s) at a weight hour space velocity (WHSV) of greater than 5 or 10 or 20 or 40 or 50 or 80 hr⁻¹, or within the range of from 5 or 8 or 10 or 20 or 40 hr⁻¹ to 300 or 1000 or 3000 or 5000 hr⁻¹; and also combined in one or more riser reactor(s) at a temperature within the range from 200 or 250 or 300 or 350° C. to 550 or 650 or 750 or 800 or 1000° C.

As used herein, “aldehyde” refers to compounds known in the art that include at least one —COH, or aldehyde, moiety. The term “aldehyde” as used herein also includes compounds and/or compositions that generate or release an aldehyde when exposed to oxygenate-to-olefins conversion conditions, such as, for example, trioxane. In one embodiment, the aldehyde is a C₁ to C₅ aldehyde such as, for example, formaldehyde, acetaldehyde, butylaldehyde, propylaldehyde, etc. In a particular embodiment, the aldehyde is formaldehyde or acetaldehyde, or compounds that generate such aldehydes. In a particular embodiment, the aldehyde is formaldehyde or a formaldehyde generating compound or composition.

In certain embodiments, the aldehyde is added in an amount to affect the desirable change in the POR for the given reactor conditions. In one embodiment, the C₁ to C₅ aldehyde is present in an amount within the range from 0.1 to 1 or 2 or 3 or 4 or 5 or 10 wt %, by weight of the methanol and aldehyde. The amount of aldehyde can be increased or decreased at a gradual rate, or stepwise. The aldehyde can be co-feed with the oxygenates, methanol in particular, or feed into the reactor through a separate line. The line may be heated or cooled, or simply insulated as is known in the art, and can inject the aldehyde at any desirable rate to achieve the desired level of aldehyde in the reactor. A diluent can be used along with the aldehyde such as, for example, air, nitrogen, water, a hydrocarbon compound, or any other suitable carrier or diluent. Thus, the weight percent values here refer to the amount of aldehyde contacted in the reactor with the molecular sieve and methanol. In particular embodiments, the on-feed lifetime of the molecular sieve is less than 40 or 30 or 20 or 15 or 10 min.

In certain embodiments, metal oxides are absent from the reactor, molecular sieve, or both during contacting with the aldehyde. As used herein, a “metal oxide,” which includes metal oxide-containing catalyst comprising an oxide of a metal, are compounds selected from the group consisting of Group 2 metals, Group 3 metals and Group 4 metals, say, an oxide of a metal selected from the group consisting of Mg, Ca, Sr, Ba and Ra, and/or an oxide of a metal selected from the group consisting of Sc, Y, La, Ce, Pr, Nd, Pm, Sm, Eu, Gd, Tb, Dy, Ho, Er, Tm, Yb, Lu, Ac, Th, Pa, U, Np, Pu, Am, Cm, Bk, Cf, Es, Fm, Md, No, and Lr, and/or an oxide of a metal selected from the group consisting of at least one of Ti, Zr, and Hf, for example, a metal oxide selected from oxides of Zr. Particular metal oxides include those oxides of the metals Sc, Y, La, and Ce. So called “Groups” refer to the Periodic Table of Elements (version published in HAWLEY'S CONDENSED CHEMICAL DICTIONARY (R. J. Lewis, 13^{th} ed., John Wiley & Sons 1997)). These compounds, and their use, are disclosed further in U.S. Pat. No. 7,205,447, herein incorporated by reference. Specific examples of metal oxides include Y₂O₃, ZnO₂, CaO₂, La₂O₃, and combinations thereof.

### Types of Molecular Sieves

Most any type of zeolitic or non-zeolitic, natural or synthetic, molecular sieve catalyst, or “molecular sieve,” can be used in the process described herein. Small and medium pore molecular sieves of structural types, such as, for example, AEI, AFI, CHA, ERI, LOV, RHO, THO, MFI and FER, are particularly useful and are well known in the art. In a particular embodiment, molecular sieves useful in the process described herein are metalloaluminophosphate molecular sieves that have a molecular framework that include [AlO₄] and [PO₄] tetrahedral units, such as metal containing aluminophosphates (AlPO). In one embodiment, the metalloaluminophosphate molecular sieves include [AlO₄], [PO₄] and [SiO₄] tetrahedral units, such as in silicoaluminophosphates (SAPO).

The molecular sieve, SAPOs in a particular embodiment, may be a single phase AEI structure type material, or may be a composition comprising an AEI structure type molecular sieve together with other structure type materials present as, for example, impurity phases, or may be a molecular sieve comprising an intergrowth of an AEI structure type material with a different structure type material, such as a CHA structure type.

Examples of single phase AEI structure type molecular sieves include SAPO-18, ALPO-18 and RUW-18. The preparation and characterization of these molecular sieves and other AEI structure type materials have been reported. Intergrown molecular sieve phases are disordered planar intergrowths of molecular sieve frameworks. In an embodiment, the molecular sieve is an intergrowth material having two or more distinct crystalline phases within one molecular sieve composition. For example, SAPO-18, AlPO-18 and RUW-18 have an AEI framework-type, and SAPO-34 has a CHA framework-type. In certain embodiments, the molecular sieve used herein may comprise two intergrowth phases of AEI and CHA framework-types, where the ratio of AEI framework-type to CHA framework-type is greater than 50 or 60 or 70 or 80 or 90%, based on the total amount of AEI and CHA structures, such as disclosed in, for example, PCT/US2009/033451. In a particular embodiment, the molecular sieves used herein contain only AEI framework type.

Desirable molecular sieves are SAPO molecular sieves, and metal-substituted SAPO molecular sieves. Suitable metal substituents are alkali metals of Group 1, an alkaline earth metals of Group 2 of the Periodic Table of Elements, a rare earth metals of Group 3, including the Lanthanides, and mixtures of any of these metal species.

In one embodiment, the metalloaluminophosphate molecular sieve is represented, on an anhydrous basis, by the formula:

mR:(MₓAl_{y}P_{z})O₂

wherein R represents at least one templating agent, preferably an organic templating agent; m is the number of moles of R per mole of (MₓAl_{y}P_{z})O₂ and m has a value from 0 to 1, preferably 0 to 0.5, and most preferably from 0 to 0.3; x, y, and z represent the mole fraction of Al, P and M as tetrahedral oxides, where M is a metal selected from the group consisting of Group 1 through 11 and Lanthanides. Preferably M is one or more metals selected from the group consisting of Si, Co, Cr, Cu, Fe, Ga, Ge, Mg, Mn, Ni, Sn, Ti, Zn and Zr. In an embodiment, m is greater than or equal to 0.2, and x, y and z are greater than or equal to 0.01. In another embodiment, m is greater than 0.1 to 1, x is greater than 0 to 0.25, y is in the range of from 0.4 to 0.5, and z is in the range of from 0.25 to 0.5.

In one embodiment, the molecular sieves useful in the disclosed process are AlPO or SAPO molecular sieves having a silica-to-alumina (“Si/Al”) ratio within a range of from 0 or 0.01 or 0.05 or 0.1 to 0.3 or 0.4 or 0.5 or 1.0 or 2.0 or 5.0 or 10.0. In a particular embodiment, the molecular sieve is a SAPO containing silicon and aluminum and having a Si/Al ratio within a range of from 0.01 or 0.05 or 0.1 to 0.3 or 0.4 or 0.5 or 1.0 or 2.0 or 5.0 or 10.0.

Non-limiting examples of SAPO and AlPO molecular sieves useful herein include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AlPO-5, AlPO-11, AlPO-18, AlPO-31, AlPO-34, AlPO-36, AlPO-37, AlPO-46, and metal containing molecular sieves thereof. Of these, particularly useful molecular sieves are one or a combination of SAPO-18, SAPO-34, SAPO-35, SAPO-44, SAPO-56, AlPO-18, AlPO-34 and metal containing derivatives thereof, such as one or a combination of SAPO-18, SAPO-34, AlPO-34, AlPO-18, and metal containing derivatives thereof.

Various methods for synthesizing molecular sieves or modifying molecular sieves are described throughout the literature. In certain embodiments, the molecular sieve, and SAPO in a particular embodiment, can have molar ratios relative to each other that are expressed in terms of their component oxides (e.g., P₂O₅, Al₂O₃, SiO₂, etc.), even though the actual source of those components may not actually be added in the oxide form or actually transform into an oxide form (for instance, the phosphorus-to-aluminum molar ratio is expressed herein as P₂O₅:Al₂O₃, even though the source of phosphorus may be added to the mixture as (aqueous) phosphoric acid, for example, and not as P₂O₅, and/or even though the source of aluminum may be added to the mixture as aluminum hydroxide (Al(OH)₃), for example, and not as Al₂O₃).

Generally, the reaction mixture formed in has a molar ratio within the following ranges:
P₂O₅:Al₂O₃ from 0.6 to 1.2,
R:Al₂O₃ from 0.5 to 2, and
H₂O:Al₂O₃ less than 30.

The final molecular sieve is typically a metalloaluminophosphate (e.g., SAPO), the reaction mixture typically having a M_{n/2}O:Al₂O₃ ratio from 0.005 to 0.6, where M is a metal, normally silicon, with a valence of n. The rate of heating employed is dependent on the H₂O:Al₂O₃ molar ratio of the reaction mixture.

The sources of the starting materials used to produce the reaction mixture are not closely controlled, but examples of suitable aluminum sources can include, though are not limited to, hydrated aluminum oxides such as boehmite and pseudoboehmite, especially pseudoboehmite. Generally, the source of phosphorus is a phosphoric acid, especially orthophosphoric acid, but other sources, for example, organic phosphates, for example, triethyl phosphate, and aluminophosphates may also be used. Where the reaction mixture includes a source of silicon, suitable silicon sources can include, but are not limited to, colloidal silica, fumed silica, and organic silica sources such as tetraalkyl orthosilicates, especially tetraethyl orthosilicate.

The organic structure directing agent can conveniently include a tetraethyl ammonium compound, such as tetraethyl ammonium hydroxide (TEAOH), tetraethyl ammonium phosphate, tetraethyl ammonium fluoride, tetraethyl ammonium bromide, tetraethyl ammonium chloride or tetraethyl ammonium acetate. In one preferred embodiment, the directing agent includes tetraethyl ammonium hydroxide. In some cases, more than one organic structure directing agent may be employed, such as a combination of a tetraethyl ammonium compound and another nitrogen-containing compound such as dipropylamine.

In certain embodiments, for example when the AEI structure type-containing molecular sieve comprises SAPO-18 (and/or AlPO-18), the organic structure directing agent can comprise one or more of an asymmetric tetraalkylammonium compound such as a triethylmethylammonium salt (e.g., a hydroxide (TEMAOH)) and a hydroxyalkylamine compound such as diisopropylaminoethanol (DIPAE). In such embodiments, the aforementioned organic structure directing agents may have significant advantages in the formation of AEI structure type-containing molecular sieves over more traditional templates such as symmetric tetraalkylammonium compounds and/or non-hydroxy-functional alkylamine compounds (e.g., TEAOH and/or diisopropylethylamine (DIPEA)). For instance, under many of the conditions where SAPO-18 (and/or AlPO-18) form(s), TEMAOH can tend to induce no significant formation of AFI and/or CHA contaminant structure type materials (and often less than with TEAOH), and DIPAE has a relatively high boiling point (e.g., 190° C., particularly as compared to DIPEA, which has a boiling point of 127° C.), which can tend to cause fewer problems in scaling up to commercial scale molecular sieve synthesis processes.

In general, molecular sieve used in commercial operations is a formulated molecular sieve. The formulated molecular sieve optionally contains binder and matrix materials. Conventionally, formulated molecular sieve is made by mixing together molecular sieve crystals (which includes template) and a liquid, optionally with matrix material and/or binder, to form a slurry. The slurry is then dried (i.e., liquid is removed), without completely removing the template from the molecular sieve. Since this dried molecular sieve includes template, it has not been activated, and is considered a preformed molecular sieve. However, the preformed molecular sieve must be activated before use by means known in the art such as calcining.

### Converting Oxygenate to Olefins Using Molecular Sieve

The molecular sieve can be contacted with oxygenate feedstock alone, or in combination with an aldehyde, converting the oxygenate to an olefin product. In a preferred embodiment of the process described herein, the oxygenate feedstock contains one or more oxygenates, more specifically, one or more organic compound(s) containing at least one oxygen atom. In the most preferred embodiment, the oxygenate in the feedstock is one or more alcohol(s), preferably aliphatic alcohol(s) where the aliphatic moiety of the alcohol(s) has from 1 to 20 carbon atoms, preferably from 1 to 10 carbon atoms, and most preferably from 1 to 4 carbon atoms. The alcohols useful as feedstock in the process described herein include lower straight and branched chain aliphatic alcohols and their unsaturated counterparts.

Non-limiting examples of oxygenates include methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof. In the most preferred embodiment, the feedstock is selected from one or more of methanol, ethanol, dimethyl ether, diethyl ether or a combination thereof, more preferably methanol and dimethyl ether, and most preferably methanol.

Desirably, the process described herein is a continuous process, wherein the molecular sieve is circulated through at least one reactor, contacted with at least methanol (and optionally aldehyde), then separated from the resulting olefins and other byproducts, optionally treated by cooling or heating, the recirculated back to at least one reactor. In certain embodiments, at least some of the molecular sieve that has been contacted with methanol (and optionally aldehyde) is directed to at least one regenerator where it can then be treated to regeneration conditions, then recirculated to at least one reactor. In this manner, a desirable level of coke can be maintained on/in the molecular sieve. In certain embodiments, the level of coke on the molecular sieve is maintained at a level within the range of from 0.5 or 1.0 or 2.0 to 5.0 or 8.0 or 10.0 or 20 wt % coke, by weight of the molecular sieve.

The olefin(s) produced from the feedstock typically have from 2 to 30 carbon atoms, preferably 2 to 8 carbon atoms, more preferably 2 to 6 carbon atoms, still more preferably 2 to 4 carbons atoms, and most preferably are ethylene and/or propylene. The reaction product can be described as a Prime Olefin Ratio, or “POR” and may be within a range of from 0.001 or 0.1 or 0.2 or 0.3 or 0.4 or 0.5 to 0.7 or 0.8 or 0.9 or 1.0 in certain embodiments.

Using the molecular sieve described herein for the conversion of a feedstock, preferably a feedstock containing one or more oxygenates, the amount of olefin(s) produced based on the total weight of hydrocarbon produced is greater than 50 wt %, typically greater than 60 wt %, such as greater than 70 wt %, and preferably greater than 75 wt %. In one embodiment, the amount of ethylene and/or propylene produced based on the total weight of hydrocarbon product produced is greater than 65 wt %, such as greater than 70 wt %, for example greater than 75 wt %, and preferably greater than 78 wt %. Typically, the amount ethylene produced in wt % based on the total weight of hydrocarbon product produced, is greater than 30 wt %, such as greater than 35 wt %, for example greater than 40 wt %. In addition, the amount of propylene produced in wt % based on the total weight of hydrocarbon product produced is greater than 20 wt %, such as greater than 25 wt %, for example greater than 30 wt %, and preferably greater than 35 wt %.

In addition to the oxygenate component, such as methanol, the feedstock may contain one or more diluent(s), which are generally non-reactive to the feedstock or molecular sieve composition and are typically used to reduce the concentration of the feedstock. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred.

The olefin conversion process can be conducted over a wide range of temperatures, such as in the range of from 200° C. to 1000° C., for example from 250° C. to 800° C., including from 250° C. to 750° C., conveniently from 300° C. to 650° C., typically from 350° C. to 600° C. and particularly from 350° C. to 550° C.

Similarly, the olefin conversion process can be conducted over a wide range of pressures including autogenous pressure. Typically the partial pressure of the feedstock exclusive of any diluent therein employed in the process is in the range of from 0.1 kPaa to 5 MPaa, such as from 5 kPaa to 1 MPaa, and conveniently from 20 kPaa to 500 kPaa.

In the olefin conversion process, the WHSV, defined as the total weight of feedstock excluding any diluents per hour per weight of molecular sieve (or molecular sieve in the molecular sieve formulation), typically ranges from greater than 5 or 10 or 20 or 40 or 50 or 80 hr⁻¹, or within the range of from 5 or 8 or 10 or 20 or 40 hr⁻¹ to 300 or 1000 or 3000 or 5000 hr⁻¹.

Where the olefin conversion process is conducted in a fluidized bed, the superficial gas velocity (SGV) of the feedstock including diluent and reaction products within the reactor system, and particularly within a riser reactor(s), is from greater than 0.1 or 0.5 or 2 or 3 or 4 or 6 or 8 or 10 meter per second (m/sec).

The process can take place in a variety of catalytic reactors such fixed bed reactors, stationary or bubbling fluidized bed reactors, turbulent fluidized bed reactors, fast fluidized bed reactors and circulating fluidized bed (“riser”) reactors, variations thereof, and combinations thereof. For example, a reactor or sections of a reactor may be a combination of a bubbling fluidized bed reactor and a riser reactor. The preferred reactor types are riser reactors generally described in FLUIDIZATION AND FLUID-PARTICLE SYSTEMS, 48-59 (F. A. Zenz and D. F. Othmo, Reinhold Publ. Corp., N.Y., 1960), and U.S. Pat. No. 6,166,282 (fast-fluidized bed reactor), which are all herein fully incorporated by reference. In one practical embodiment, the process is conducted in at least one high velocity process, utilizing one or more risers, at least one regeneration system and a recovery system.

In the olefin conversion process, the reactor system preferably includes a reactor system having a first reaction zone within one or more riser reactor(s) and a second reaction zone within at least one disengaging vessel, typically comprising one or more cyclones. In one embodiment, the one or more riser reactor(s) and disengaging vessel are contained within a single reactor vessel. Fresh feedstock, preferably containing one or more oxygenates, optionally with one or more diluent(s), is fed to the one or more riser reactor(s) into which a molecular sieve composition or coked version thereof is introduced. In one embodiment, regenerated or fresh, uncoked molecular sieve, prior to being introduced to the riser reactor(s), is pretreated with the olefin composition described herein, and then contacted with oxygenate feedstock to convert the oxygenate to olefin product at high selectivity to ethylene and propylene.

In another embodiment, oxygenate feedstock is fed to the olefin conversion reactor as a liquid and/or a vapor in a range of from 0.1 wt % to 99.9 wt %, such as from 1 wt % to 99 wt %, more typically from 5 wt % to 95 wt %, based on the total weight of the feedstock including any diluent contained therein. The liquid and vapor feedstocks may be the same composition, or may contain varying proportions of the same or different feedstocks with the same or different diluents.

The feedstock entering the reactor system is preferably converted, partially or fully, in the first reactor zone into a gaseous effluent that enters the disengaging vessel along with the coked molecular sieve. After combining the molecular sieve and oxygenate and optionally the aldehyde, the level of coke on the molecular sieve in certain embodiments is within the range of from 0.01 or 0.1 or 1 wt % to 2 or 3 or 5 or 10 wt %, by weight of the molecular sieve. In the preferred embodiment, cyclone(s) are provided within the disengaging vessel to separate the coked molecular sieve from the gaseous effluent containing one or more olefins) within the disengaging vessel. Although cyclones are preferred, gravity effects within the disengaging vessel can also be used to separate the molecular sieve from the gaseous effluent. Other methods for separating the molecular sieve composition from the gaseous effluent include the use of plates, caps, elbows, and the like.

When present, the aldehyde is added as a mixture with the oxygenate to contact the molecular sieve, or injected at the same time as the oxygenate but in a different location in the reactor. Preferably, the aldehyde is contacted with the molecular sieve under the same, or only slightly different, conditions as the conditions for the oxygenate-molecular sieve contacting. Thus, the aldehyde is added in the same step as the oxygenate is added. Preferably, pretreatment of the molecular sieve with aldehyde, in the absence of oxygenate, is not performed. That is, preferably, the molecular sieve is not contacted with added aldehyde alone and without simultaneous contacting with an oxygenate such as methanol.

In one embodiment, the disengaging vessel includes a stripping zone, typically in a lower portion of the disengaging vessel. In the stripping zone the coked molecular sieve is contacted with a gas, preferably one or a combination of steam, methane, carbon dioxide, carbon monoxide, hydrogen, or an inert gas such as argon, preferably steam, to recover adsorbed hydrocarbons from the coked molecular sieve that is then introduced to the regeneration system.

The “coked” molecular sieve is withdrawn from the disengaging vessel and introduced to the regeneration system. The regeneration system comprises a regenerator where the coked molecular sieve is contacted with a regeneration medium, preferably a gas containing oxygen, under conventional regeneration conditions of temperature, pressure and residence time. Non-limiting examples of suitable regeneration media include one or more of oxygen, O₃, SO₃, N₂O, NO, NO₂, N₂O₅, air, air diluted with nitrogen or carbon dioxide, oxygen and water, carbon monoxide and/or hydrogen. Suitable regeneration conditions are those capable of burning coke from the coked molecular sieve, preferably to a level less than 0.5 wt % based on the total weight of the coked molecular sieve composition entering the regeneration system. For example, the regeneration temperature may be in the range of from 200° C. to 1500° C. The regeneration pressure may be in the range of from 15 psia (103 kPaa) to 500 psia (3448 kPaa).

The residence time of the molecular sieve in the regenerator may be in the range of from one minute to several hours, such as from one minute to 100 minutes. The amount of oxygen in the regeneration flue gas (i.e., gas which leaves the regenerator) may be in the range of from 0.01 mol % to 5 mol % based on the total volume of the gas. The amount of oxygen in the gas used to regenerate the coked molecular sieve (i.e., fresh or feed gas) is typically at least 15 mol %, based on total amount of regeneration gas fed to the regenerator.

In one embodiment, the regenerated molecular sieve is withdrawn from the regeneration system and combined with a fresh molecular sieve composition and/or re-circulated molecular sieve composition and/or feedstock and/or fresh gas or liquids, and returned to the riser reactor(s). A carrier, such as an inert gas, feedstock vapor, steam or the like, may be used, semi-continuously or continuously, to facilitate the introduction of the regenerated molecular sieve to the reactor system, preferably to the one or more riser reactor(s).

Gaseous effluent is withdrawn from the disengaging system and is passed through a recovery system to recover product and by-products, including the olefin pretreatment composition. Conventional recovery systems, techniques and sequences can be used in separating olefin(s) and purifying olefin(s) from the gaseous effluent. Conventional recovery systems generally comprise one or more or a combination of various separation, fractionation and/or distillation towers, columns, splitters, or trains, reaction systems such as ethylbenzene manufacture and other derivative processes such as aldehydes, ketones and ester manufacture, and other associated equipment, for example various condensers, heat exchangers, refrigeration systems or chill trains, compressors, knock-out drums or pots, pumps, and the like. Non-limiting examples of these towers, columns, splitters or trains used alone or in combination include one or more of a demethanizer, preferably a high temperature demethanizer, a dethanizer, a depropanizer, a wash tower often referred to as a caustic wash tower and/or quench tower, absorbers, adsorbers, membranes, ethylene (C₂^{═}) splitter, propylene (C₃^{═}) splitter and butene (C₄^{═}) splitter.

Typically, in converting one or more oxygenates to ethylene and/or propylene, a minor amount hydrocarbon, particularly olefin(s), having 4 or more carbon atoms is also produced. The amount of C₄+ olefins is normally less than 20 wt %, such as less than 10 wt %, for example less than 5 wt %, and particularly less than 2 wt %, based on the total weight of the effluent gas withdrawn from the process, excluding water. Typically, therefore the recovery system may include one or more reaction systems for converting the C₄+ olefins to useful products.

In one practical embodiment, the olefin forming process is integrated with one or more polyolefin processes to produce any one of a variety of polyolefins. In such an integrated process, at least one olefin in the olefin product stream, preferably ethylene or propylene, is separated and contacted with a polymerization catalyst to form a polyolefin product (e.g., polyethylene or polypropylene). The polyolefin product can be further treated as desired or shipped to other destinations for further treatment or processing.

Polymerization processes that can be integrated with the olefin conversion processes useful herein include solution, gas phase, slurry phase, high pressure processes, and combinations thereof. Particularly preferred is a gas phase or a slurry phase polymerization of one or more olefin(s) at least one of which is ethylene or propylene. These polymerization processes utilize a polymerization catalyst that can include any one or a combination of the molecular sieves discussed above. However, the preferred polymerization catalysts are the Ziegler-Natta, Phillips-type, metallocene, metallocene-type and advanced polymerization catalysts, and mixtures thereof.

In a preferred embodiment, the integrated process comprises a process for polymerizing one or more olefin(s) in the presence of a polymerization catalyst system in a polymerization reactor to produce one or more polymer products, wherein the one or more olefin(s) have been made by converting an alcohol, particularly methanol, using a molecular sieve composition as described above. The preferred polymerization process is a gas phase polymerization process and at least one of the olefins(s) is either ethylene or propylene, and preferably the polymerization catalyst system is a supported metallocene catalyst system. In this embodiment, the supported metallocene catalyst system comprises a support, a metallocene or metallocene-type compound and an activator, preferably the activator is a non-coordinating anion or alumoxane, or combination thereof, and most preferably the activator is alumoxane.

The polymers produced by the polymerization processes described above include linear low density polyethylene, elastomers, plastomers, high density polyethylene, low density polyethylene, polypropylene and polypropylene copolymers. The propylene based polymers produced by the polymerization processes include atactic polypropylene, isotactic polypropylene, syndiotactic polypropylene, and propylene random, block or impact copolymers.

Thus, disclosed in a particular embodiment is a method of producing ethylene comprising combining in a reactor methanol and a SAPO having a Si/Al ratio within the range of from 0.05 to 2.0 under conditions to produce at least ethylene and propylene having a first ethylene/propylene ratio, adding to the reactor within the range of from 0.01 to 2.0 wt % of a C₁ to C₅ aldehyde, by weight of the aldehyde and methanol, and withdrawing from the reactor a first amount of ethylene and propylene having a second ethylene/propylene ratio, wherein the second ethylene/propylene ratio is greater than the first ethylene/propylene ratio, wherein the methanol, molecular sieve and aldehyde are combined in one or more riser reactor(s) at a WHSV of greater than 5 hr⁻¹. The ethylene that is produced is then isolated through standard separation means to isolate ethylene from ethane, propane, propylene, and other typical side produces such as dimethyl ether, etc. The ethylene and propylene, ethylene in particular, can then be combined with an olefin polymerization catalyst to produce polyethylene homopolymers and copolymers (when also combined with, for example, propylene, hexene, vinyl acetate, and/or other polymerizable monomers).

### Examples

SAPO-18 samples were synthesized according to PCT/US2009/033451. The Si/Al ratio was 0.130 for the SAPO-18 used in these experiments.

The olefins synthesis reaction was carried out on a fixed-bed microreactor. Methanol was fed at a preset pressure and rate to stainless steel reactor tube housed in an isothermally heated zone. The reactor tube contained 20 mg weighed and sized granules of the molecular sieve sample (20-40 mesh by press-and-screen method). The molecular sieve SAPO-18 was activated for 30 minutes at 500° C. in flowing nitrogen before methanol or methanol/trioxane mixture was admitted. The product effluent was sampled at different times during the run with a twelve-port sampling loop while the molecular sieve was continuously deactivating. The effluent sample in each port was analyzed with a Gas Chromatograph equipped with a flame ionization detector.

The testing condition was as follows: the temperature was 475° C. and the pressure of methanol was 275 kPaa. The feed rate in weight hourly space velocity (“WHSV”) was 100/h. Cumulative conversion of methanol was expressed as grams of methanol converted per gram of molecular sieve (“CMCPS”, the number of grams of methanol converted from the initial (100%) conversion to the point of 10% methanol conversion). On-stream lifetime refers to the CMCPS when methanol conversion has dropped to 10%. The product selectivity was reported as averages over the entire conversion range, rather than from a single point in effluent composition.

Data demonstrating the olefin production performance of the molecular sieve with and without trioxane (which decomposes to formaldehyde upon heating) are shown in Tables 1 and 2. As the results indicate, on-feed lifetime was reduced with increasing amount of trioxane feed, and the Prime Olefin Ratio (“POR”) increased. The level of coke in the molecular sieve was measured by temperature program oxidation.

### Temperature Programmed-Oxidation (TPO)

TPO (temperature programmed-oxidation) was used to measure the amount of carbon during coke-removing experiments. TPO was carried out by loading 5 to 10 mg of a MTO molecular sieve in a quartz reactor. A carrier gas containing 1% O₂ in helium was introduced to the quartz reactor at a rate of 63 ml/minute. The reactor was heated at a constant rate of 13° C. per minute. The gas exiting the quartz reactor was directed to a methanator, which contained a ruthenium catalyst. This catalyst was operated at 400° C. to convert CO and CO₂ to methane. The methane produced was continuously measured with a flame ionization detector (FID). To measure the CO production during the TPO experiments, a CO₂ trap filled with ascarite (sodium hydroxide impregnated solids) was used to completely remove CO₂ from the exit gas before it was sent to the methanator. Therefore, FID acquired with the ascarite trap exclusively measured the amount of CO that was produced from the oxidation of carbon on the molecular sieve. Details of this technique are described by S. C. Fung and C. A. Querini in 138 J. CAT. 240 (1992) and C. A. Querini and S. C. Fung in 141 J. CAT. 389 (1993), both incorporated herein by reference.

TABLE 1

| | g MeOH Converted/g | | | C₂⁼ + | |
|---|---|---|---|---|---|
| | molecular sieve @ X = 10% | Total g MeOH | C₂⁼/C₃⁼ | C₃⁼ | Initial |
| Run Description | (on-feed life time) | Converted | (POR) | (POS) | Conversion |
| Pure methanol feed | 40.87 | 33.4 | 0.56 | 71.2 | 99.5% |
| 0.5 wt % trioxane in methanol | 10.85 | 12.8 | 0.60 | 69.8 | 99.4% |
| 1 wt % trioxane in methanol | 9.18 | 10.4 | 0.62 | 68.4 | 98.5% |

TABLE 2

| | | | | | | | | | Coke |
|---|---|---|---|---|---|---|---|---|---|
| Run Description | CH₄ | C2⁼ | C2 | C3⁼ | C3 | C4's | C5's | C6+'s | (wt %) |
| Pure methanol feed | 1.2 | 25.7 | 0.2 | 45.5 | 0.4 | 19.7 | 5.0 | 0.7 | 1.5 |
| 0.5 wt % trioxane in methanol | 2.1 | 26.3 | 0.4 | 43.5 | 0.9 | 18.1 | 4.8 | 0.8 | 3.1 |
| 1 wt % trioxane in methanol | 3.3 | 26.2 | 0.4 | 42.1 | 0.9 | 17.3 | 4.7 | 0.8 | 4.2 |

Having described the various aspects of the oxygenates-to-olefins process, and the process to produce olefins, especially ethylene, described herein in numbered embodiments is:
1. A method of controlling production of olefins in an oxygenates-to-olefins reaction comprising:
   combining in a reactor methanol and a molecular sieve under conditions to produce at least ethylene and propylene having a first ethylene/propylene ratio;
   adding to the reactor a first amount of a C₁ to C₅ aldehyde; and
   withdrawing from the reactor a first amount of ethylene and propylene having a second ethylene/propylene ratio, wherein the second ethylene/propylene ratio is greater than the first ethylene/propylene ratio.
2. The method of numbered embodiment 1, wherein metal oxides are substantially absent from the reactor and molecular sieve.
3. The method of numbered embodiments 1 and 2, wherein the addition is performed in one step at the same reactor conditions as the combining step.
4. The method of any one of the previous numbered embodiments, wherein the molecular sieve is an aluminophosphate or a silicoaluminophosphate.
5. The method of numbered embodiment 4, wherein the silcoaluminophosphate is selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, metal containing molecular sieves thereof, and combinations thereof
6. The method of any one of the previous numbered embodiments, wherein the C₁ to C₅ aldehyde is present in an amount within the range from 0.1 to 1 or 2 or 3 or 4 or 10 wt %, by weight of the methanol and aldehyde.
7. The method of any one of the previous numbered embodiments, wherein the methanol, molecular sieve and aldehyde are combined in one or more riser reactor(s) at a WHSV of greater than 5 or 10 or 20 or 40 or 50 or 80 hr⁻¹.
8. The method of any one of the previous numbered embodiments, wherein the methanol, molecular sieve and aldehyde are combined in one or more riser reactor(s) at a temperature within the range from 200 or 250 or 300 or 350° C. to 550 or 650 or 750 or 800 or 1000° C.
9. The method of any one of the previous numbered embodiments, wherein after combining, the level of coke on the molecular sieve is within the range of from 0.01 or 0.1 or 1 wt % to 2 or 3 or 5 or 10 wt %, by weight of the molecular sieve.
10. The method of any one of the previous numbered embodiments, wherein the C₁ to C₅ aldehyde is selected from formaldehyde and acetaldehyde.
11. The method of any one of the previous numbered embodiments, wherein the molecular sieve, having been combined with methanol and optionally an aldehyde is separated from the olefin product and directed to a regeneration step.
12. The method of numbered embodiment 11, wherein the level of coke on the molecular sieve is maintained at a level within the range of from 0.5 or 1.0 or 2.0 to 5.0 or 8.0 or 10.0 wt % coke, by weight of the molecular sieve.
13. The method of any one of the previous numbered embodiments, adding to the reactor a third amount of a C₁ to C₅ aldehyde, wherein the third amount of aldehyde is greater than the first amount of aldehyde; withdrawing from the reactor a second amount of ethylene and propylene having a second ethylene/propylene ratio, wherein the second ethylene/propylene ratio is greater than the first ethylene/propylene ratio.
14. The method of any one of the previous numbered embodiments, wherein the on-feed lifetime is less than 40 or 30 or 20 or 15 or 10 min
15. The method of any one of the previous numbered embodiments, wherein the molecular sieve possesses a Si/Al ratio within the range of from 0 or 0.01 or 0.05 or 0.1 to 0.3 or 0.4 or 0.5 or 1.0 or 2.0 or 5.0 or 10.0.
16. The method of any one of the previous numbered embodiments, further comprising combining the ethylene and/or propylene with an olefin polymerization catalyst to produce a polyolefin.
17. A method of producing ethylene comprising:
   combining in a reactor methanol and a SAPO having a Si/Al ratio within the range of from 0.05 or 0.1 to 0.2 or 0.3 or 0.5 or 1.0 or 2.0 under conditions to produce at least ethylene and propylene having a first ethylene/propylene ratio;
   adding to the reactor within the range of from 0.01 or 0.1 or 0.2 to 0.8 or 1.0 or 1.5 or 2.0 wt % of a C₁ to C₅ aldehyde, by weight of the aldehyde and methanol; and
   withdrawing from the reactor a first amount of ethylene and propylene having a second ethylene/propylene ratio, wherein the second ethylene/propylene ratio is greater than the first ethylene/propylene ratio;
   wherein the methanol, molecular sieve and aldehyde are combined in one or more riser reactor(s) at a WHSV of greater than 5 or 10 or 20 or 40 or 50 or 80 hr⁻¹.
18. The method of numbered embodiment 17, wherein the methanol, molecular sieve and aldehyde are combined in one or more riser reactor(s) at a temperature within the range from 200 or 250 or 300 or 350° C. to 550 or 650 or 750 or 800 or 1000° C.
19. The method of any one of the previous numbered embodiments 17-18, wherein after combining, the level of coke on the molecular sieve is within the range of from 0.01 or 0.1 or 1 wt % to 2 or 3 or 5 or 10 wt %, by weight of the molecular sieve.
20. The method of any one of the previous numbered embodiments 17-19, wherein the C₁ to C₅ aldehyde is selected from formaldehyde and acetaldehyde.
21. The method of any one of the previous numbered embodiments 17-20, wherein the on-feed lifetime is less than 40 or 30 or 20 or 15 or 10 min.
22. The method of any one of the previous numbered embodiments 17-21, wherein metal oxides are substantially absent from the reactor and SAPO.
23. The method of any one of the previous numbered embodiments 17-22, wherein the addition is performed in one step at the same reactor conditions as the combining step.
24. The method of any one of the previous numbered embodiments 17-23, wherein the ratio of AEI framework-type to CHA framework-type in the SAPO is greater than 50 or 60 or 70 or 80 or 90%, based on the total amount of AEI and CHA structures in the SAPO.
25. The method of any one of the previous numbered embodiments 17-24, further comprising combining the ethylene and/or propylene with an olefin polymerization catalyst to produce a polyolefin.

What is claimed is:

1. A method of controlling production of olefins in an oxygenates-to-olefins reaction comprising:
combining in a reactor methanol and a molecular sieve under conditions to produce at least ethylene and propylene having a first ethylene/propylene ratio;
adding to the reactor a first amount of a C₁ to C₅ aldehyde; and
withdrawing from the reactor a first amount of ethylene and propylene having a second ethylene/propylene ratio, wherein the second ethylene/propylene ratio is greater than the first ethylene/propylene ratio.

2. The method of claim 1, wherein metal oxides are substantially absent from the reactor and molecular sieve.

3. The method of claim 1, wherein the addition is performed in one step at the same reactor conditions as the combining step.

4. The method of claim 1, wherein the molecular sieve is an aluminophosphate or a silicoaluminophosphate.

5. The method of claim 4, wherein the silcoaluminophosphate is selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, metal containing molecular sieves thereof, and combinations thereof.

6. The method of claim 1, wherein the C₁ to C₅ aldehyde is present in an amount within the range from 0.1 to 10 wt %, by weight of the methanol and aldehyde.

7. The method of claim 1, wherein the methanol, molecular sieve and aldehyde are combined in one or more riser reactor(s) at a WHSV of greater than 5 hr⁻¹.

8. The method of claim 1, wherein the methanol, molecular sieve and aldehyde are combined in one or more riser reactor(s) at a temperature within the range from 200° C. to 1000° C.

9. The method of claim 1, wherein after combining, the level of coke on the molecular sieve is within the range of from 0.01 wt % to 10 wt %, by weight of the molecular sieve.

10. The method of claim 1, wherein the C₁ to C₅ aldehyde is selected from formaldehyde and acetaldehyde.

11. The method of claim 1, wherein the molecular sieve, having been combined with methanol and optionally an aldehyde is separated from the olefin product and directed to a regeneration step.

12. The method of claim 11, wherein the level of coke on the molecular sieve is maintained at a level within the range of from 0.5 to 10.0 wt % coke, by weight of the molecular sieve.

13. The method of claim 1, wherein the amount of C₁ to C₅ aldehyde added to the reactor is within the range of from 0.01 to 2.0 wt %, by weight of the C₁ to C₅ aldehyde and methanol.

14. The method of claim 1, wherein the on-feed lifetime is less than 40 min.

15. The method of claim 1, wherein the molecular sieve possesses a Si/Al ratio within the range of from 0 to 10.

16. The method of claim 1, further comprising combining the ethylene and/or propylene with an olefin polymerization catalyst to produce a polyolefin.

17. A method of producing ethylene comprising:
combining in a reactor methanol and a SAPO having a Si/Al ratio within the range of from 0.05 to 2.0 under conditions to produce at least ethylene and propylene having a first ethylene/propylene ratio;
adding to the reactor within the range of from 0.01 to 2.0 wt % of a C₁ to C₅ aldehyde, by weight of the aldehyde and methanol; and
withdrawing from the reactor a first amount of ethylene and propylene having a second ethylene/propylene ratio, wherein the second ethylene/propylene ratio is greater than the first ethylene/propylene ratio;
wherein the methanol, molecular sieve and aldehyde are combined in one or more riser reactor(s) at a WHSV of greater than 5 hr⁻¹.

18. The method of claim 17, wherein the methanol, molecular sieve and aldehyde are combined in one or more riser reactor(s) at a temperature within the range from 200° C. to 1000° C.

19. The method of claim 17, wherein after combining, the level of coke on the molecular sieve is within the range of from 0.01 wt % to 10 wt %, by weight of the molecular sieve.

20. The method of claim 17, wherein the C₁ to C₅ aldehyde is selected from formaldehyde and acetaldehyde.

21. The method of claim 17, wherein the on-feed lifetime is less than 40 min.

22. The method of claim 17, wherein metal oxides are substantially absent from the reactor and SAPO.

23. The method of claim 17, wherein the addition is performed in one step at the same reactor conditions as the combining step.

24. The method of claim 17, wherein the ratio of AEI framework-type to CHA framework-type in the SAPO is greater than 50%, based on the total amount of AEI and CHA structures in the SAPO.

25. The method of claim 17, further comprising combining the ethylene and/or propylene with an olefin polymerization catalyst to produce a polyolefin.
